(19) United States
(12) Patent Application Publication
(10) Pub. No.: US 2023/0293218 A1
(43) Pub. Date: Sep. 21, 2023

(54) APPARATUSES AND METHODS FOR MONITORING AND CONTROLLING BLEEDING DURING CRYOABLATION TREATMENTS

(71) Applicant: Varian Medical Systems, Inc., Palo Alto, CA (US)

(72) Inventor: Hongxuan Zhang, Austin, TX (US)

(21) Appl. No.: 17/655,218

(22) Filed: Mar. 17, 2022

(51) Int. Cl. A61B 18/02

(52) U.S. Cl. CPC A61B 18/02; A61B 2018/00875

(57) ABSTRACT

A system to perform cryoablation treatments includes at least one computing device configured to obtain at least one impedance measurement from a cryoprobe and determine whether a bleeding condition is present at a treatment site proximate the cryoprobe based on the at least one impedance measurement. The at least one computing device also adjusts one or more inputs to a heater in the cryoprobe when the at least one impedance measurements indicate that the bleeding condition is present.

### FIELD

The present disclosure relates to apparatuses and methods for monitoring and controlling bleeding during cryoablation treatments.

### BACKGROUND

This section provides background information related to the present disclosure which is not necessarily prior art.

Systems and methods for providing cryoablation treatments may include cryoablation probes that are introduced at or near target tissue in a patient. A cryoablation system may include an extremely cold cryo-fluid (liquid, gas, or mixed phase) that may be passed through a probe in thermal contact with the target tissue. Heat from the tissue passes from the tissue, through the probe, and into the fluid that removes heat from the targeted tissue. This removal of heat causes tissue to freeze, resulting in the destruction of the targeted tissue. The cryo-fluid may also be heated subsequent to the freezing cycle. The heating may thaw the frozen tissue to allow the cryoprobe to be removed from the tissue. The heating may also be used to coagulate blood in instances of bleeding at the cryoablation site. It is desirable to reduce or minimize bleeding that may occur during cryoablation treatments to minimize the impact of the bleeding to the health of the patient.

Traditional or existing systems and methods do not include elements or methods of determining whether a bleeding condition has occurred and/or to effectively or efficiency address bleeding conditions should they be present. In addition, differences between treatment sites, patients, tissue, and other factors can make it difficult for existing and traditional systems to reduce or minimize the impact of bleeding during cryoablation treatments. Still further, the elements of traditional and existing cryoablation systems do not include advantageous equipment to allow bleeding conditions to be monitored, controlled or remediated. There exists a need, therefore, for improved cryoablation systems and methods to detect, monitor and control system parameters to efficiently and effectively reduce or minimize bleeding conditions that may occur during the course of treatment.

### SUMMARY

This section provides a general summary of the disclosure, and is not a comprehensive disclosure of its full scope or all of its features.

In some embodiments, methods and systems for monitoring and controlling bleeding conditions during cryoablation treatments are provided. In one example, a cryoablation system may include a computing device coupled to a cryoprobe. One or more impedance measurements from the cryoprobe may be obtained by the computing device. The computing device may determine based on the impedance measurements whether a bleeding condition is present in the patient. If the a bleeding condition is present, the computing device may initiate a coagulation cycle in the cryoprobe to raise the temperature of the tissue at the site of the bleeding condition. The computing device may monitor the bleeding condition by comparing the impedance measurements or a rate of change of the impedance measurements to predetermined thresholds or to a predicted impedance path. Once the bleeding condition has been addressed, the cryoablation treatment may continue.

In some embodiments, a system to perform cryoablation treatments may include at least one computing device. The computing device is configured to obtain at least one impedance measurement from a cryoprobe and determine whether a bleeding condition is present at a treatment site proximate the cryoprobe based on the at least one impedance measurement. The computing device is also configured to adjust one or more inputs to a heater in the cryoprobe when the at least one impedance measurements indicate that the bleeding condition is present.

In one aspect, the cryoprobe may include an impedance sensor coupled to the at least one computing device.

In another aspect, the impedance sensor may be located on an external surface of the cryoprobe.

In another aspect, the at least one computing device may determine whether a bleeding condition is present by comparing the at least one impedance measurement to an impedance threshold.

In another aspect, the at least one computing device may be further configured to determine a bleeding level associated with the bleeding condition by comparing the at least one impedance measurement to one or more impedance ranges.

In another aspect, the at least one computing device may be further configured to obtain a treatment plan comprising tissue information and to determine whether the bleeding condition is present further based on the tissue information.

In another aspect, the one or more inputs may include at least one of a current, a frequency, a power profile and a voltage.

In another aspect, the at least one impedance measurement may include an external impedance measurement and an internal impedance measurement.

In another aspect, the at least one computing device may determine a temperature of the cryoprobe based on the internal impedance measurement.

In another aspect, the at least one computing device obtains the internal impedance measurement from the heater in the cryoprobe.

In another aspect, the at least one computing device obtains a plurality of impedance measurements and determines that the bleeding condition is present based on a rate of change of the plurality of impedance measurements.

In another aspect, the at least one computing device may obtain a plurality of impedance measurements and a predicted impedance profile, and compares the plurality of impedance measurements to the predicted impedance profile.

In some embodiments, a method of performing a cryoablation treatment is provided. The method may include obtaining at least one impedance measurement from a cryoprobe and determining whether a bleeding condition is present at a treatment site proximate the cryoprobe based on the at least one impedance measurement. The method may also include adjusting one or more inputs to a heater in the cryoprobe when the at least one impedance measurements indicate that the bleeding condition is present.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is diagram illustrating an example cryoablation system in accordance with some embodiments of the present disclosure.

FIG. 2 is another diagram illustrating another example cryoablation system in accordance with some embodiments of the present disclosure.

FIG. 3 is a flow chart illustrating an example method of performing a cryoablation treatment that includes a coagulation cycle in accordance with some embodiments of the present disclosure.

FIG. 4 is a flow chart illustrating an example coagulation method in accordance with some embodiments of the present disclosure.

FIG. 5 is a graph illustrating an impedance value over time during an example coagulation cycle.

FIG. 6 is a side view of an example cryoprobe of the present disclosure.

FIG. 7 is a flow chart illustrating an example coagulation method in accordance with some embodiments of the present disclosure.

FIG. 8 is a diagram illustrating an example computing device that can be used in the cryoablation systems of the present disclosure.

Corresponding reference numerals indicate corresponding parts throughout the several views of the drawings.

### DETAILED DESCRIPTION

Example embodiments will now be described more fully with reference to the accompanying drawings.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

In various embodiments of the present disclosure, improved cryoablation systems and methods are provided. The cryoablation systems and methods of the present disclosure can detect whether a bleeding condition is present during a cryoablation treatment using one or more impedance measurements obtained from the cryoprobe. Various control devices of the cryoablation system can adjust various settings of the cryoprobe in response to determining that a bleeding condition is present.

The cryoablation system may, for example, initiate a coagulation cycle. In such a cycle the temperature of the tissue at the bleeding condition is raised to a predetermined temperature to allow coagulation to occur. The cryoablation system may continue to monitor and control the setting of the system, such as the heater in the cryoprobe, to promote coagulation. This action can reduce and/or minimize harm that may be caused by a bleeding condition in the patient.

The systems and methods are improvements over existing and traditional systems. Rather than relying on external systems or experience of a medical professional, the cryoablation systems and methods of the present disclosure include bleeding control and monitoring in addition to providing traditional cryoablation capability. Furthermore, the cryoablation systems and methods of the present disclosure can efficiently and effectively monitor and control bleeding conditions during cryoablation treatments. Still further, the cryoablation systems and methods of the present disclosure provide smart closed-loop systems for real time controlling of coagulation cycles during treatment.

The cryoablation systems of the present disclosure may also use one or more elements or methods as described in U.S. patent application Ser. No. TBD entitled “APPARATUSES AND METHODS FOR ADAPTIVELY CONTROLLING CRYOABLATION SYSTEMS” filed on the same day as the present application by Varian Medical Systems, Inc., U.S. patent application Ser. No. TBD entitled “APPARATUSES AND METHODS FOR SEQUENTIAL HEATING OF CRYO-FLUID IN CRYOABLATION SYSTEMS” filed on the same day as the present application by Varian Medical Systems, Inc., and U.S. patent application Ser. No. TBD entitled “APPARATUSES AND METHODS FOR THE CONTROL AND OPTIMIZATION OF ICE FORMATION DURING CYROABLATION TREATMENTS” filed on the same day as the present application by Varian Medical Systems, Inc., the disclosures of which are hereby incorporated by reference in their entireties.

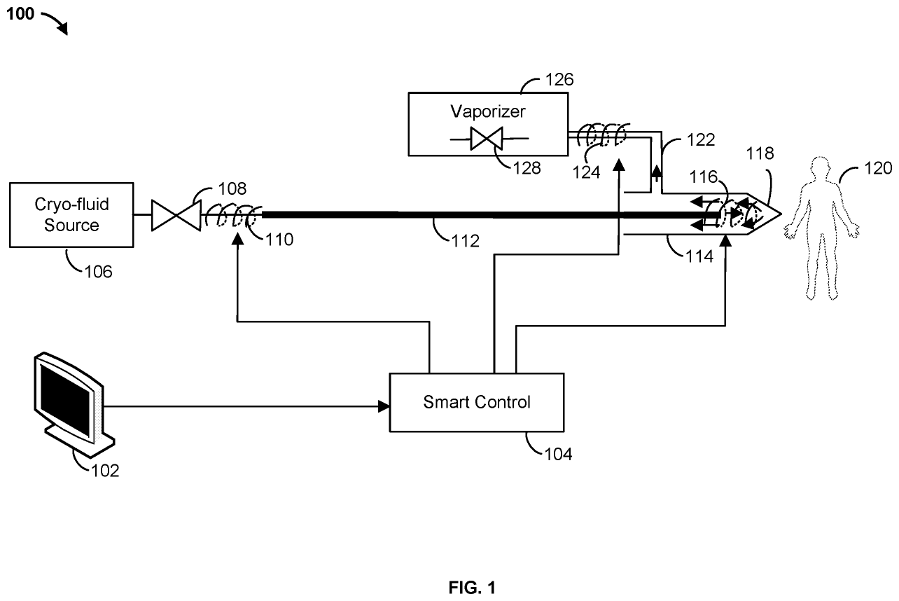

Turning now to FIG. 1, an example cryoablation system 100 is shown. The cryoablation system 100 may include a cryoablation computing device 102, a smart control 104, a cryo-fluid source 106, an inlet valve 108, a first heater 110, a cryo-fluid supply 112, a cryoprobe 114, a vaporizer 126, a second heater 124, an exhaust valve 128, and a cryo-fluid return 122. The cryo-fluid source 106, the inlet valve 108, the first heater 110, the cryo-fluid supply 112, the cryoprobe 114, the vaporizer 126, the exhaust valve 128, and the cryo-fluid return 122 may operate to deliver a cryo-fluid from the cryo-fluid source 106 to the cryoprobe 114 to perform a cryoablation treatment. The cryo-fluid (e.g., liquid nitrogen) can be stored in the cryo-fluid source 106, such as a dewar or other suitable container, and then delivered to the cryoprobe 1114 via the cryo-fluid supply 112. The cryo-fluid may expand at a tip 118 of the cryoprobe 114 and cool the tip 118 of the cryoprobe 114 to a temperature at which the tissue of a patient 120 begins to freeze forming an iceball.

The cryoprobe 114 can be positioned at or near a target tissue (e.g., a tumor) in the patient. In this manner, the target tissue can be frozen destroying the target tissue. Once a freezing cycle is complete, a thaw cycle can be initiated. The thaw cycle can be used so that the cryoprobe 114 can be extracted. The thaw cycle can also stop the iceball from continuing to form and/or prevent damage to healthy tissues that may located near to or surrounding the target tissue. In still other examples, the thaw cycle may be used to coagulate or otherwise reduce or stop bleeding that may occur at or near the cryoablation site. The cryoprobe 114 may be heated to a temperature that causes the coagulation of blood to occur (e.g., at or above 100° F. or at or above 110° F.). During a thaw cycle or a coagulation cycle, a probe heater 116 may be used to heat the cryo-fluid and/or the cryoprobe 114. The cryo-fluid may be evacuated from the cryoprobe 114 to allow the cryoprobe to warm. The cryo-fluid may flow from the cryoprobe 114 through the cryo-fluid return 122 and be vaporized by the vaporizer 126 and/or exhausted to the environment via the exhaust valve 128. During various cryoablation treatments, one or more freezing, coagulation and/or thaw cycles may be used.

A treatment plan can be determined prior to the performance of the cryoablation treatment. The treatment plan can detail and/or describe the various steps of the process and various aspects of the treatment such as the types of equipment to be used, a positioning of the cryoprobe, temperatures of the cryoprobe, duration of freezing and thaw cycles as well as a quantity of cycles. The treatment plan may be determined by a medical professional and/or by others. In some examples, the cryoablation computing device 102 may determine or recommend a treatment plan after health, patient, and other information is input into the cryoablation computing device 102 or is retrieved or otherwise obtained by the cryoablation computing device 102.

In traditional or existing systems and methods, it can be difficult to determine whether a bleeding condition is present during a cryoablation treatment. The cryoprobe 114, in this example, may include multiple impedance sensors or measuring devices. The impedance measurements from these impedance sensors can be used to determine whether a bleeding condition is present and a level of bleeding that may be present.

As shown, the cryoablation system 100 may also include a smart control 104 and a cryoablation computing device 102. The smart control 104 may be coupled to the cryoablation computing device 102 and to the first heater 110, the second heater 124 and the cryoprobe 114. The smart control 104 can be any suitable controller, PLC, data acquisition unit or other control unit. The smart control 104 is operable not only to receive impedance and/or other measurement signals from the other elements of the cryoablation system 100 but may also be operable to control, change or adjust operating parameters of the cryoablation system 100. For example, the smart control 104 may be operable to control the power inputs of the first heater 110, the second heater 124 and/or the probe heater 116. As will be further described, the smart control can change, adjust or control the voltage, current, power profile, frequency and timing of the power delivered to the first heater 110, the second heater 124 and/or the probe heater 116. Such control can be used, for example, to manage coagulation cycles of the cryoablation system 100.

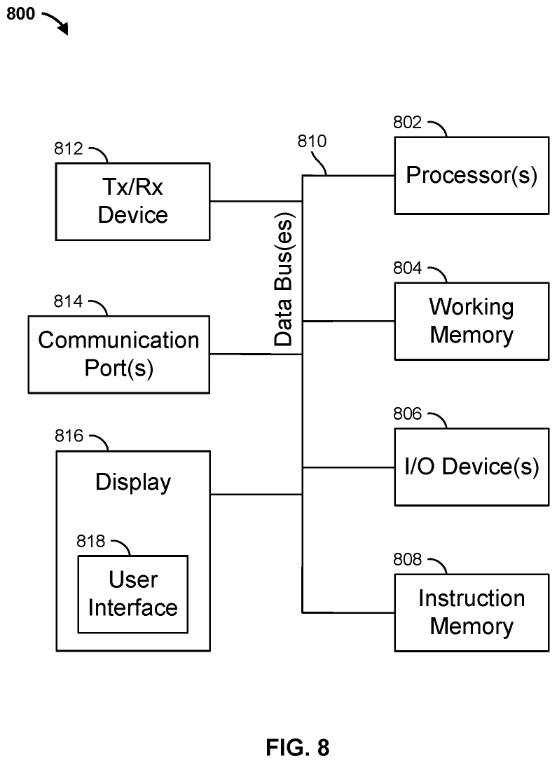

The cryoablation computing device 102 can be any suitable computing device that can operate to receive and process data and provide instructions to the smart control 104. The cryoablation computing device may be, for example, a suitable workstation, computer, laptop, tablet, server or the like. In some example, the cryoablation computing device may have the structure shown (and described below) in FIG. 8.

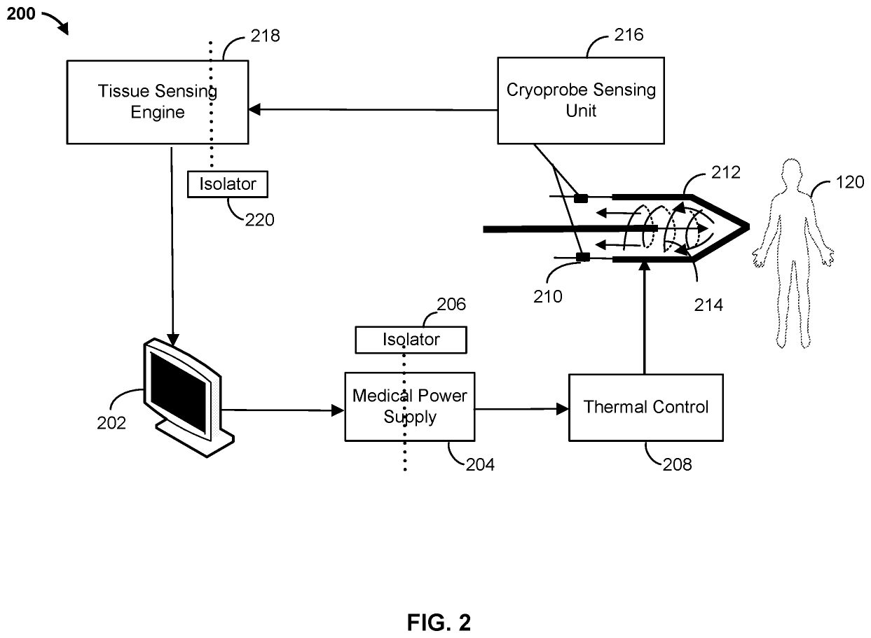

Another example cryoablation system 200 is shown in FIG. 2. The cryoablation system 200 is similar in many respects to the cryoablation system 100 previously described. In this example, the cryoablation system 200 includes a cryoablation computing device 202 coupled to a medical power supply 204 and a thermal control 208. The thermal control 208 is, in turn, coupled to the cryoprobe 212. This portion of the cryoablation system 100 allows the cryoablation computing device 202 to provide instructions for the medical power supply 204 and the thermal control 208 to energize the probe heater 214 as desired to perform a controlled coagulation cycle. The medical power supply 204 may be any suitable medical-grade power supply that can provide electrical energy to the cryoprobe heater 214. The electrical energy from the medical power supply 204 can be provided through the thermal control 208. The thermal control 208 can be any suitable power transformer that can provide variable electrical energy that may vary the voltage, frequency, waveform, power profile, current and the like to the probe heater 214. The instructions for the power profile or other power parameters may be provided by the cryoablation computing device 202, for example.

As shown, the cryoablation computing device is also coupled to a tissue sensing engine 218, and a cryoprobe sensing unit 216. The cryoprobe sensing unit 216 may be coupled to one or more sensors or other measurement structures in the cryoprobe 212. As shown the cryoprobe 212 may include one or more impedance sensors 210 on an external surface of the cryoprobe 212. The cryoprobe sensing unit 216 may also be coupled to the cryoprobe heater 214. The cryoprobe heater 214 may be configured as a coil heater. The cryoprobe sensing unit 216 may also obtain an impedance measurement from the coil heater.

The cryoprobe sensing unit 216 may be any suitable data acquisition unit, computing device or the like that can receive signals from the impedance sensors and/or impedance measurement devices of the cryoprobe 212. The signals may be converted to impedance measurements and sent or provided to the tissue sensing engine 218. The tissue sensing engine may be a suitable processing device, application or software module that can determine whether a bleeding condition is present. The tissue sensing engine may receive or obtain information from the cryoablation computing device 202 or other suitable information source regarding the treatment plan for the patient. The treatment plan may include information regarding the patient, the target tissue or other information regarding the cryoablation site. This information may be used in conjunction with the impedance information to determine whether a bleeding condition exists.

Information regarding the impedance measurements, treatment plan, patient information and the like may be obtained or received by the cryoablation computing device 202. The cryoablation computing device can both monitor the status and possible bleeding conditions of the patient 120 while also controlling a coagulation cycle of the cryoprobe 212.

As further shown, isolators 206 and 220 may positioned at the medical power supply 204 and the tissue sensing engine 218, respectively. Isolators 206 and 220 may be isolators as described in a testing standard such as the International Electrotechnical Commission (IEC) IEC60601 standard that requires patient and medical equipment isolation between main power to patient and floating ground (GND). The isolators are for patient and equipment current leakage (such as earth, enclosure, etc.). The cryoablation system may be classified as a Body Floating (BF) part for patient current requirements. The isolators 206, 220 may be a transformer or medical graded power supply that is certified by an IEC testing facility.

While FIG. 2 shows the various elements of the cryoablation system 100 separately, it should be appreciated that one or more of the elements may be combined. For example, the tissue sensing engine 218, the cryoprobe sensing unit 216, and/or the thermal control 208 may be combined in one or more computing devices. In some examples, these elements are combined as part of cryoablation computing device 102. In other examples, other combinations can be used.

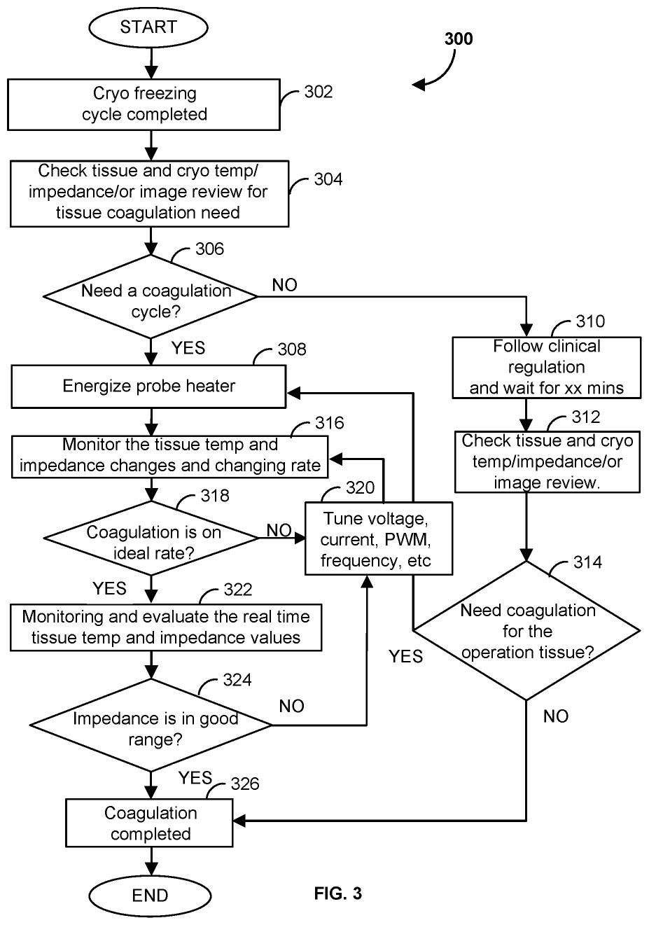

Referring now to FIG. 3, an example method 300 of performing a coagulation cycle is shown. The method 300 may be performed during or in connection with a cryoablation treatment. The method 300 may be performed by various cryoablation apparatuses or systems of the present disclosure. For example, the method 300 may be performed by the cryoablation system 100 or 200 previously described. The description below describes the method 300 relative to the cryoablation system 200 but it should be appreciated that other systems and apparatuses can also be used.

At step 302, the cryo freezing cycle of a cryoablation treatment has been completed. At step 304, the cryoablation computing device 202 may determine if a coagulation cycle is needed. The cryoablation computing device 202 may receive impedance measurements or may receive information from the tissue sensing engine 218 via the cryoprobe sensing unit. This information can be compared to various predetermined thresholds, impedance profiles and/or other conversion methods.

In some examples, the impedance measurements from the external impedance sensors 210 of the cryoprobe 212 are compared to one or more impedance thresholds or impedance ranges. Using experimental or historical data, the impedance thresholds or impedance ranges can be determined that indicate that bleeding condition is present. For example, it can be determined that an impedance of the external surface sensor 210 of the cryoprobe 212 will change in the presence of blood. In one example, it has been determined that in a dry condition (i.e., in the absence of blood) the impedance of the cryoprobe surface sensor 210 will be approximately 800 ohms. In the presence of a mild bleeding condition, the impedance of the cryoprobe surface sensor 210 may decrease to a value in the range of about 300 ohms to 600 ohms. In the presence of a significant bleeding condition, the impedance of the cryoprobe surface sensor 210 may decrease to a value in the range of about 150 ohms or less.

In this example, the impedance measurement of the surface sensor 210 may be compared to the predetermined thresholds or ranges to determine whether a bleeding condition is present and/or a level of the bleeding condition. In the example described above, if an impedance measurement of 800 ohms is obtained, the cryoablation computing device 202 and/or the tissue sensing engine 218 may determine that no bleeding condition is present and that no coagulation cycle is needed. If the impedance measurement has a value of 100 ohms. The cryoablation computing device 202 or the tissue sensing engine 218 may determine that a significant bleeding condition is present and may determine that a coagulation cycle is needed.

In yet other examples, and as will be further described, the cryoablation computing device 202 and/or the tissue sensing engine 218 may determine whether a coagulation cycle is present by comparing the impedance values or a rate of change of the impedance values to an expected impedance profile. The predetermined impedence thresholds, the predetermined impedance ranges and/or the expected impedance thresholds may be different for different patients, for different target tissues or for different conditions for the cryoablation treatment.

The predetermined impedance thresholds, the predetermined impedance ranges and/or the expected impedance profile may be retrieved by the cryoablation computing device 202 and/or the tissue sensing engine 218 from a database or health information source. In other examples, the thresholds, the ranges and/or expected impedance profile may be entered by a user via a user interface of the cryoablation computing device 202.

In still further examples, the cryoablation computing device 102 and/or the tissue sensing engine 218 may obtain other health information such as imaging information from an imaging device such as an ultrasound device, x-ray device, CT scan device, or the like. The imaging data or other health information may be used by the cryoablation computing device 102 and/or the tissue sensing engine 218 to determine if a coagulation cycle is required.

If the cryoablation computing device 202 determines that no coagulation cycle is needed, the method 300 proceeds to step 310. If the cryoablation computing device 202 determines that a coagulation cycle is needed, the method 300 proceeds to step 308.

At step 308, the cryoablation computing device 202 may energize the probe heater 214. The cryoablation computing device 202 may, for example, send instructions via the medical power supply 204 and/or the thermal control 208 to provide power to the probe heater 214. The instruction may include a particular power profile or specify the current, voltage, frequency, pulse width or other information regarding the power to be delivered to the probe heater 214. The power profile may be determined, for example, based on a level of bleeding that may be present. In other examples, the power profile may be determined based on the type of tissue in which the cryoablation treatment is being performed. In yet other examples, the power profile may be pre-set to have certain levels by a user or medical professional.

At step 316, various conditions of the cryoablation site are monitored by the cryoablation computing device 202. The temperature of the tissue as measured by the impedance of the probe heater can be monitored. The cryoablation computing device 202 may also monitor the impedance at the surface of the cryoprobe at the external sensor 210. These measurement may be obtained, recorded and stored over time to determine a rate of change of these measurements. The cryoablation computing device 202 may, for example record, store and display the measurement as a graph over time. As such, the rate of change or slope of the curve for these measurements can be monitored by the cryoablation computing device 202.

At step 318, the cryoablation computing device 202 may determine whether the coagulation is proceeding in an expected or desirable manner. One way of determining this is by comparing the impedance values to impedance thresholds as previously described. Another way of determining a progress of coagulation is to compare the rate of change of the impedance value to an expected rate of change. If coagulation is progressing in a desirable manner, the impedance value should be increasing. The rate of change of the impedance can be compared to the expected rate of change to determine whether further action is needed to improve the coagulation process. If the cryoablation computing device 202 determines that coagulation is proceeding in a desirable manner, the method 300 proceeds to step 322. If the cryoablation computing device 202 determines that coagulation is not proceeding in a desirable or ideal manner, the method 300 may proceed to step 320.

At step 320, the cryoablation computing device 202 may take action to improve the coagulation process. The cryoablation computing device 202 may modify one or more settings or parameters of the cryoablation system 200. The cryoablation computing device 202 may change the power being delivered to the probe heater 214. If coagulation is performing slower than desired (i.e., the bleeding condition is not improving quickly enough), the temperature may be increased to promote coagulation. In other circumstances, the cryoablation computing device 202 may determine that coagulation is improving quickly enough but there is a risk of burning or otherwise causing harm to tissue based on an elevated temperature of the cryoprobe 212. In these circumstances, the cryoablation computing device 202 may instruct the thermal control 208 to reduce the power to the probe heater 214. Based on the circumstances and the measurements, the cryoablation computing device may take action and change the operating conditions of the cryoablation system to improve efficiency, improve coagulation and/or to reduce or minimize harm to healthy tissues of the patient.

After taking such action to improve the operation of the cryoablation system 200, the method 300 returns to step 308. The cryoablation computing device 202 may perform the steps as previously described until such time that the cryoablation computing device 202 determines that coagulation is progressing in a desirable manner and/or at a desired or ideal rate.

At step 322, the cryoablation computing device 202 may continue to monitor the various measurements obtained from the impedance sensors 210 and/or from the probe heater 214. The cryoablation computing device 202 can measure and monitor the impedance and temperature of the tissue at the cryoablation site. In this manner, the cryoablation computing device 202 can monitor the temperature of the tissue and determine a status and level of the bleeding condition.

At step 324, the cryoablation computing device 202 can determine whether the impedance value is in a preferred range. As previously described, the cryoablation computing device 202 can compare the impedance value to one or more predetermined impedance thresholds or to one or more predetermined impedance ranges. The coagulation cycle may continue, for example, until the bleeding condition is not detected. The cryoablation computing device 202 may determine that no bleeding is occuring when the impedance value exceeds a certain value (e.g., 800 ohms). In other examples, other thresholds or values can be used depending on the tissue, patient and other factors.

If the cryoablation computing device 202 determines that the impedance value is not in a good range or does not exceed a predetermined threshold, the method 300 can return to step 320. The cryoablation computing device 202 can take action to correct, control or improve the coagulation process at step 320 as previously described. If the cryoablation computing device 202 determines that the impedance value is in a preferred range or exceeds the predetermined impedance threshold, the method 300 can proceed to step 326 at which the coagulation cycle is completed.

If the cryoablation computing device 202 determined that a coagulation cycle was not needed at step 306, the method 300 proceeds to step 310. At step 310, the cryoablation computing device 202 can follow the treatment plan and wait for a predetermined waiting time. The cryoablation computing device 202 may obtain a treatment plan that describes or includes clinical regulations and other predetermined aspects of the cryoablation treatment. Clinical regulations may, for example, include predetermined waiting times that are used to determine whether bleeding conditions or other circumstances arise during the course of a cryoablation treatments.

After waiting the predetermined waiting time, the method 300 may proceed to step 312. At step 312, the cryoablation computing device 202 may obtain impedance and temperature measurements as described above with respect to step 304. The cryoablation computing device 202 may then again determine whether a coagulation cycle is needed at step 314. Step 314 may be performed similarly to step 306 previously described. If the cryoablation computing device 202 determines that a coagulation cycle is needed, the method proceeds to step 308 and continues as previously described. If the cryoablation computing device 202 determines that a coagulation cycle is not needed, the method proceeds to steps 326 and the coagulation cycle is complete.

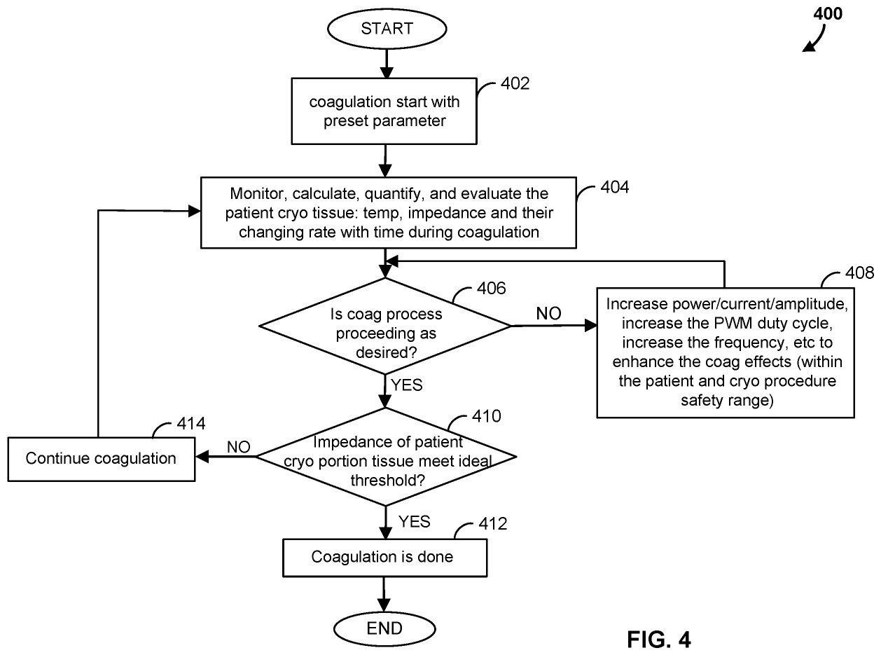

Another example method of performing a coagulation cycle is shown in FIG. 4. The method 400 may be performed during or in connection with a cryoablation treatment. The method 400 may be performed by various cryoablation apparatuses or systems of the present disclosure. For example, the method 400 may be performed by the cryoablation system 100 or 200 previously described. The description below describes the method 300 relative to the cryoablation system 200 but it should be appreciated that other systems and apparatuses can also be used.

At step 402, the cryoablation computing device 202 may start with one or more preset parameters or other operational settings. The preset parameters may be obtained as part of a treatment plan. The preset parameters may be obtained from a database or other health information system, for example. The preset parameters may include, for example, power profiles for one or more of the heaters, impedance thresholds, impedance ranges, impedance profiles and the like. At step 402, the cryoablation computing device 202 may energize the probe heater 214 using the preset power profile. As such, the cryoprobe 212 may begin warming as a result.

At step 404, the cryoablation computing device 202 may monitor, calculate, quantify and evaluate the cryo tissue. Such operations may be performing based on the temperature, impedance and other information obtained by the cryoablation computing device 202. As discussed with method 300, the cryoablation computing device may obtain impedance measurements from impedance sensors 210 and temperature information from the probe heater 214. In other examples, other sensors and measurement devices may provide information to the cryoablation computing device 202.

At step 406, the cryoablation computing device 202 may determine whether the coag process is proceeding as desired. As the tissue in the patient at the cryoablation site warms, the coagulation process should improve and the bleeding condition should improve. At step 406, the cryoablation computing device 202 may compare the impedance, temperature or other measurements to predetermined thresholds, predetermined ranges, and/or predetermined profiles to determine the status of the coagulation process and whether action is required to improve the coagulation process.

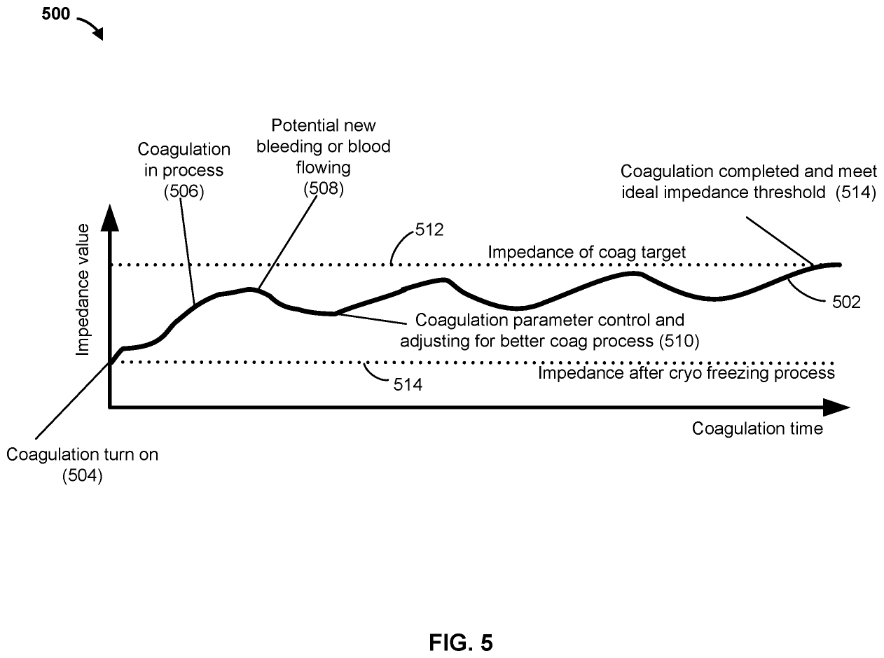

In some examples, the cryoablation computing device 202 may compare the impedance measurements from the impedance sensors 210 to an impedance profile. In other examples, the cryoablation computing device 202 may compare a rate of change of the impedance measurements to predetermined thresholds or to predetermined coagulation rules. An example is shown in FIG. 5. FIG. 5 shows an example graph of impedance measurement over time. The line 502 tracks the impedance measurements measured by, for example, the impedance sensors 210. As shown, at location 504, the coagulation cycle starts when the probe heater 214 is energized. As can be seen, the impedance value is approximately equal to the lower threshold 514 corresponding to an impedance value after the cryo freezing cycle.

As coagulation cycle progresses, the line 502 shows that the impedance value increases and the coagulation cycle is in process at region 506. The increasing impedance value suggests that a bleeding condition may be improving. At region 508 of the line 502, the impedance value begins to decrease. The change in slop of the line at region 508 and a negative rate of change may suggest that the bleeding condition is no longer improving and blood flow may be increasing or a new bleeding condition may be present. When the cryoablation computing device 202 determines that such a condition is occurring, by reviewing the rate of change of the impedance value, for example, the cryoablation computing device 202 can adjust or change one or more operating parameters of the cryoablation computing device.

The cryoablation computing device 202 may continue to monitor the impedance value and control the operating parameters of the cryoablation computing device to keep the impedance value flowing a positive slope with a positive rate of change. When the cryoablation computing device 202 determines that the rate of change is the impedance value is decreasing and/or becomes negative, the cryoablation computing device may change or adjust one or more operating parameters such as increasing or changing a power level delivered to the probe heater 214. The cryoablation computing device 202 may continue to monitor, control and adjust as necessary until the impedance value is equal to or greater than a predetermined impedance threshold or coagulation target 512. At location 514, the impedance value has reached the impedance threshold 512.

Referring back to method 400, the cryoablation computing device 202 may monitor the impedance, temperature and other measurements to determine if the coagulation cycle is proceeding as desired at step 406. If the cryoablation computing device 202 determines that the coagulation process is proceeding as desired, the method 400 may proceed to step 410. If the cryoablation computing device 202 determines that the cryoablation computing device is not proceeding as desire, the method 400 may proceed to step 408.

At step 408, the cryoablation computing device 202 may take action to improve the coagulation process. In some examples, the cryoablation computing device 202 may increase the power, current, amplitude or other setting of the cryoablation system 200. In other examples, the cryoablation computing device 202 may increase the PWM duty cycle, increase the frequency or otherwise adjust the power profile being delivered to the probe heater 214. As can be appreciated, such adjustments and changes may be limited to prescribed safety or other levels. Such limits may be included in the treatment or otherwise entered by a user or medical professional.

At step 410, the cryoablation computing device 202 may determine whether the impedance has met or exceeded a predetermined impedance threshold. Such threshold, for example, may correspond to a level that indicates the bleeding condition has improved or is no longer present. If the cryoablation computing device 202 determines that the impedance has reached or exceeded the predetermined impedance threshold, the method 400 may proceed to step 412 at which time the coagulation cycle is complete. If the cryoablation computing device 202 determines that the impedance value is not at or greater than the predetermined impedance threshold, the method may proceed to step 414 and return to step 404. The cryoablation computing device 202 may continue to monitor the impedance, temperature and other measurements as previously described and take action to improve the coagulation process if necessary.

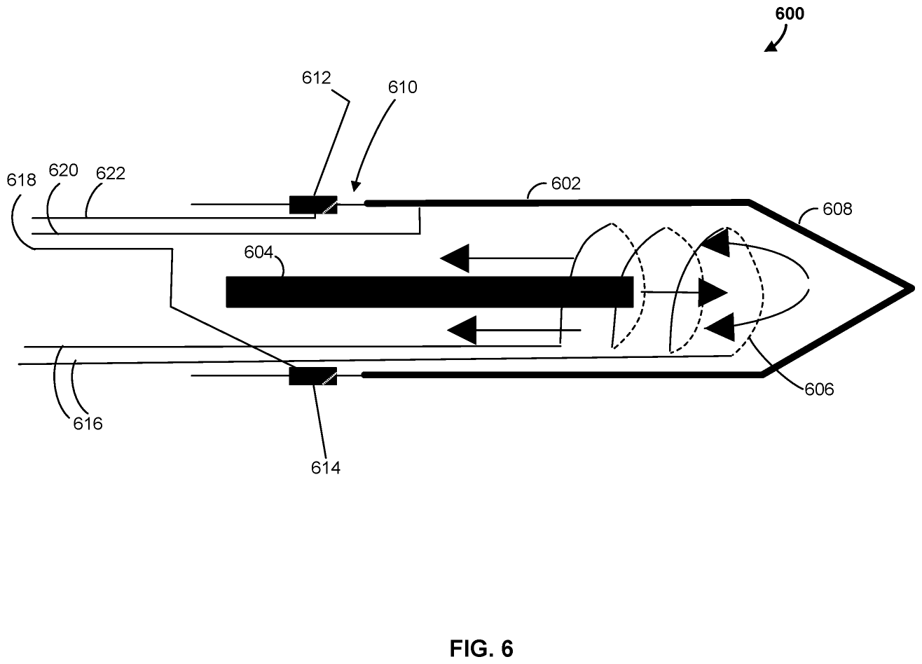

Turning now to FIG. 6, another example cryoprobe 600 is shown. The cryoprobe 600 can be used in the cryoablation systems 100, 200 previously described and may be used in connection with the methods of the present disclosure. The cryoprobe 600 may include an outer shell 602 that terminates at tip 608. The shell 602 may be made of suitable metal material material. The tip 608 may function as a measurement lead. The tip signal line 620 may be coupled to the tip 608 can be configured to deliver an impedance signal to the cryoablation computing device 202 via the cryoprobe sensing unit 216.

The cryoprobe 600 may also include other impedance sensors positioned at different locations along the shell 602. The cryoprobe 600 may include, for example, external impedance sensors 612, 614 positioned at an axial location away from the tip 608. The impedance sensors 612, 614 may be insulated from the tip 608 by an insulating layer, ring, or other insulating section 610. In this manner, the impedance sensors 612, 614 can provide independent impedance measurements from that provided by the tip 608. The impedance sensor 612, 614 can be configured as a ring, band or other strip of metal material on the cryoprobe 600. The impedance sensors 612, 614 can be coupled to the cryoablation computing device 202 via the cryoprobe sensing unit 216 by the second impedance signal line 622, and the third impedance signal line 618, respectively.

In other examples, the cryoprobe 600 may include more impedance sensors than that shown or previously described. The impedance sensors can be positioned at any axial or radial positions on the cryoprobe 600. In this manner, further information indicating a location of a bleeding condition can be collected. It may desirable, for example, to include impedance sensors at locations spaced apart from one another at predetermined radial positions. Such impedance sensors can be positioned at 90 degree intervals around a circumference of the cryoprobe 600. The impedance sensors will include further insulating sections to electrically insulate the impedance sensors from each other. The insulating sections can be portions of ceramic, plastic or other materials that may surround or be positioned between neighboring impedance sensors.

As further shown, the cryoprobe 600 includes one or more temperature sensors. The cryoprobe 600 may utilize the heating coil 606 not only to heat the cryoprobe 600 but also to collect temperature measurements of the cryoprobe and surrounding tissue. The coil leads 616 may be coupled to the cryoprobe sensing unit 216 and/or to the cryoablation computing device 202 to deliver the temperature measurement signal from the cryoprobe 600. In other examples, the cryoprobe 600 may include other temperature sensors at other locations on the cryoprobe 600.

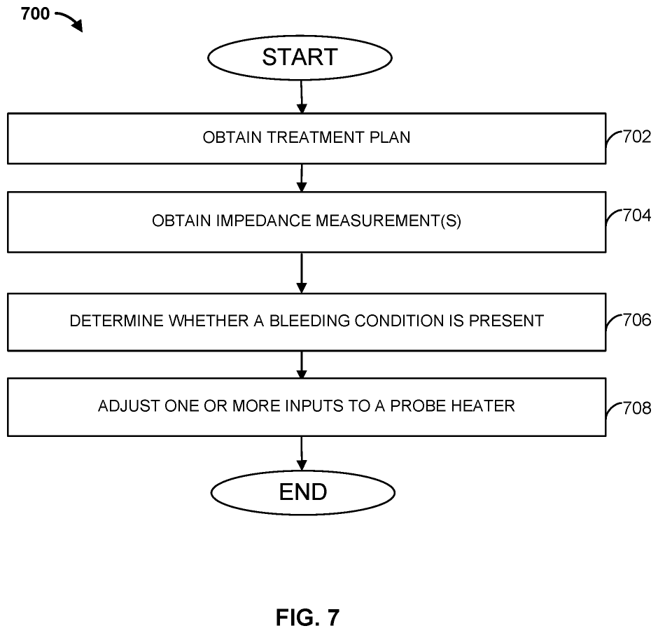

Turning now to FIG. 7, another example method 700 of performing a coagulation cycle is shown. The method 700 may be performed during or in connection with a cryoablation treatment. The method 700 may be performed by various cryoablation apparatuses or systems of the present disclosure. For example, the method 400 may be performed by the cryoablation system 100 or 200 previously described. The description below describes the method 700 relative to the cryoablation system 200 but it should be appreciated that other systems and apparatuses can also be used.

At step 702, the cryoablation computing device 202 may obtain a treatment plan. The treatment plan may include various aspects, settings, procedures, thresholds, limits, ranges and other information that describes the cryoablation treatment. The treatment plan can be obtained by the cryoablation computing device 202 by retrieving the treatment plan from a database, health information system or other storage device. In other examples, the treatment plan can be input into the cryoablation computing device 202 by a user or medical professional using a user interface.

At step 704, the cryoablation computing device 202 may obtain impedance measurements. The impedance measurements can be obtained using the cryoprobes previously described. The impedance measurements may be obtained by the cryoablation computing device 202 via the cryoprobe sensing unit 216, for example.

At step 706, the cryoablation computing device may determine whether a bleeding condition is present. The cryoablation computing device 202 may compare the impedance measurement to predetermined thresholds, to predetermined ranges, and/or to predetermined profiles. The cryoablation computing device 202 may also determine whether a bleeding condition is present by determining a rate of change of the impedance. The cryoablation computing device 202 may determine whether a bleeding condition is present using one or more of the methods previously described.

In some examples, the cryoablation computing device 202 may also determine a severity level of the bleeding condition. The cryoablation computing device 202 may determine the severity level, for example, by comparing the impedance measurements to predetermined impedance ranges. The ranges may determined using historical or laboratory data. The predetermined impedance ranges may be different between patients, between tissues, and/or between other conditions.

At step 708, the cryoablation computing device 202 may adjust one or more inputs to a probe heater in the cryoprobe when the cryoablation computing device 202 determines that a bleeding condition is present. The cryoablation computing device 202 may, for example, energize the probe heater. In other circumstances, the cryoablation computing device 202 may adjust or change at least one of a current, voltage, frequency, pulse width, power profile or other parameter provided to the probe heater.

As can be appreciated, in some circumstances, the steps 704 through 708 can be repeated and continuously or periodically performed to control bleeding conditions that may occur during a cryoablation treatment. These steps may be performed until such time that the cryoablation computing device 202 determines that no bleeding conditions are present.

Referring now to FIG. 8, an example computing device 800 is shown. The cryoablation system 100 or 200 may include one or more computing devices 800. For example, the cryoablation computing device 102 or 202 may have the elements shown in FIG. 8. The methods of the present disclosure, such as methods 300, 400, and 700, may be performed, or steps of such methods may be performed, by a computing device 800.

As shown, the computing device 800 may include one or more processors 802, working memory 804, one or more input/output devices 806, instruction memory 808, a transceiver 812, one or more communication ports 814, and a display 816, all operatively coupled to one or more data buses 810. Data buses 810 allow for communication among the various devices. Data buses 810 can include wired, or wireless, communication channels.

Processors 802 can include one or more distinct processors, each having one or more cores. Each of the distinct processors can have the same or different structure. Processors 802 can include one or more central processing units (CPUs), one or more graphics processing units (GPUs), application specific integrated circuits (ASICs), digital signal processors (DSPs), and the like.

Processors 802 can be configured to perform a certain function or operation by executing code, stored on instruction memory 808, embodying the function or operation. For example, processors 802 can be configured to perform one or more of any function, step, method, or operation disclosed herein.

Instruction memory 808 can store instructions that can be accessed (e.g., read) and executed by processors 802. For example, instruction memory 808 can be a non-transitory, computer-readable storage medium such as a read-only memory (ROM), an electrically erasable programmable read-only memory (EEPROM), flash memory, a removable disk, CD-ROM, any non-volatile memory, or any other suitable memory.

Processors 802 can store data to, and read data from, working memory 804. For example, processors 802 can store a working set of instructions to working memory 804, such as instructions loaded from instruction memory 808. Processors 802 can also use working memory 804 to store dynamic data created during the operation of cryoablation computing device 102 or 202. Working memory 804 can be a random access memory (RAM) such as a static random access memory (SRAM) or dynamic random access memory (DRAM), or any other suitable memory.

Input-output devices 806 can include any suitable device that allows for data input or output. For example, input-output devices 806 can include one or more of a keyboard, a touchpad, a mouse, a stylus, a touchscreen, a physical button, a speaker, a microphone, or any other suitable input or output device.

Communication port(s) 814 can include, for example, a serial port such as a universal asynchronous receiver/transmitter (UART) connection, a Universal Serial Bus (USB) connection, or any other suitable communication port or connection. In some examples, communication port(s) 814 allows for the programming of executable instructions in instruction memory 808. In some examples, communication port(s) 814 allow for the transfer (e.g., uploading or downloading) of data.

Display 816 can display a user interface 818. User interfaces 818 can enable user interaction with the cryoablation computing device 102 or 202. For example, user interface 818 can be a user interface that allows an operator to interact, communicate, control and/or modify different messages, settings, or features that may be presented or otherwise displayed to a user. The user interface 818 can include a slider bar, dialogue box, or other input field that allows the user to control, communicate or modify a setting, limitation or input that is used in a cryoablation treatment. In addition, the user interface 818 can include one or more input fields or controls that allow a user to modify or control optional features or customizable aspects of the cryoablation computing device 102 or 202 and/or the operating parameters of the cryoablation system 100 or 200. In some examples, a user can interact with user interface 818 by engaging input-output devices 806. In some examples, display 816 can be a touchscreen, where user interface 818 is displayed on the touchscreen. In other examples, display 816 can be a computer display that can be interacted with using a mouse or keyboard.

Transceiver 812 allows for communication with a network. In some examples, transceiver 812 is selected based on the type of communication network cryoablation computing device 102 or 202 will be operating in. Processor(s) 802 is operable to receive data from, or send data to, a network, such as wired or wireless network that couples the elements of the cryoablation system 100 or 200.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A system to perform cryoablation treatments comprises at least one computing device, the at least one computing device configured to:
obtain at least one impedance measurement from a cryoprobe;
determine whether a bleeding condition is present at a treatment site proximate the cryoprobe based on the at least one impedance measurement; and
adjust one or more inputs to a heater in the cryoprobe when the at least one impedance measurements indicate that the bleeding condition is present.

2. The system of claim 1, further comprising the cryoprobe including an impedance sensor coupled to the at least one computing device.

3. The system of claim 2, wherein the impedance sensor is located on an external surface of the cryoprobe.

4. The system of claim 1, wherein the at least one computing device determines whether a bleeding condition is present by comparing the at least one impedance measurement to an impedance threshold.

5. The system of claim 1, wherein the at least one computing device is further configured to determine a bleeding level associated with the bleeding condition by comparing the at least one impedance measurement to one or more impedance ranges.

6. The system of claim 1, wherein the at least one computing device is further configured to obtain a treatment plan comprising tissue information and to determine whether the bleeding condition is present further based on the tissue information.

7. The system of claim 1, wherein the one or more inputs comprises at least one of a current, a frequency, a power profile and a voltage.

8. The system of claim 1, wherein the at least one impedance measurement comprises an external impedance measurement and an internal impedance measurement.

9. The system of claim 8, wherein the at least one computing device determines a temperature of the cryoprobe based on the internal impedance measurement.

10. The system of claim 9, wherein the at least one computing device obtains the internal impedance measurement from the heater in the cryoprobe.

11. The system of claim 1, wherein the at least one computing device obtains a plurality of impedance measurements and determines that the bleeding condition is present based on a rate of change of the plurality of impedance measurements.

12. The system of claim 1, wherein the at least one computing device obtains a plurality of impedance measurements and a predicted impedance profile, and compares the plurality of impedance measurements to the predicted impedance profile.

13. A method of performing a cryoablation treatment comprising:
obtaining at least one impedance measurement from a cryoprobe;
determining whether a bleeding condition is present at a treatment site proximate the cryoprobe based on the at least one impedance measurement; and
adjusting one or more inputs to a heater in the cryoprobe when the at least one impedance measurements indicate that the bleeding condition is present.

14. The method of claim 13, wherein the cryoprobe comprises an impedance sensor coupled to at least one computing device.

15. The method of claim 14, wherein the impedance sensor is located on an external surface of the cryoprobe.

16. The method of claim 13, wherein the step of determining whether a bleeding condition is present is performed by comparing the at least one impedance measurement to an impedance threshold.

17. The method of claim 13, wherein the step of determining the bleeding level associated with the bleeding condition is performed by comparing the at least one impedance measurement to one or more impedance ranges.

18. The method of claim 13, further comprising obtaining a treatment plan comprising tissue information and determining whether the bleeding condition is present further based on the tissue information.

19. The method of claim 13, wherein the one or more inputs comprises at least one of a current, a frequency, a power profile and a voltage.

20. The method of claim 13, wherein the at least one impedance measurement comprises an external impedance measurement and an internal impedance measurement.

21. The method of claim 20, further comprising determining a temperature of the cryoprobe based on the internal impedance measurement.

22. The method of claim 21, wherein the internal impedance measurement is obtained from the heater in the cryoprobe.

23. The method of claim 13, wherein the at least one impedance measurement comprises a plurality of impedance measurements and the method further comprises determining that the bleeding condition is present based on a rate of change of the plurality of impedance measurements.

24. The method of claim 13, wherein further comprising obtaining a predicted impedance profile and comparing the at least one impedance measurement to the predicted impedance profile.
